(19) United States
(12) Patent Application Publication
(10) Pub. No.: US 2023/0096950 A1
(43) Pub. Date: Mar. 30, 2023

(54) METHOD FOR AUTOMATIC ACTUATION AND OVERALL SYSTEM

(71) Applicant: Siemens Healthcare GmbH, Erlangen (DE)

(72) Inventors: Marcus Pfister, Bubenreuth (DE); Markus Kowarschik, Nürnberg (DE); Tim Horz, Nürnberg (DE)

(21) Appl. No.: 17/955,917

(22) Filed: Sep. 29, 2022

(30) Foreign Application Priority Data
Sep. 30, 2021 (DE) 10 2021 210 983.7

(51) Int. Cl. G16H 40/63

(52) U.S. Cl. CPC G16H 40/63

(57) ABSTRACT

A method for automatic actuation of at least one device in the medical environment including recording of at least one image in a medical environment with an arrangement in which at least one person of the medical staff and at least one device that is adjustable or movable by motorized mechanisms are present, segmentation of the image and identification of the at least one person and/or the at least one adjustable or movable device, evaluation of the arrangement in the image in terms of at least one ergonomic parameter with regard to the at least one person, determination of an adjustment or a movement of the at least one device, which improves the ergonomic parameter with regard to the at least one person, and actuation of the device for autonomous adjustment or movement of the device such that the ergonomic parameter is improved.

### CROSS REFERENCES TO RELATED APPLICATIONS

This application claims the benefit of DE 10 2021 210 983.7 filed on Sep. 30, 2021, which is hereby incorporated by reference in its entirety.

### FIELD

Embodiments relate to a method for automatic actuation of at least one device in the medical environment.

### BACKGROUND

As a result of advances in technology, operating techniques, and development of instruments, more and more complex medical procedures are being carried out in operating theaters and special clinics, due to which the sequences of the procedures are also becoming increasingly complex. Examples of this include for example minimally invasive operations using imaging devices such as C-arm X-ray devices for example. As a result, the physical requirements on the staff involved are also increasing for various reasons. The increasing length of operations, which the staff mostly take part in while standing and with a heavy lead apron, has a negative effect on the posture and spinal column of staff. Additionally, many operations require a large number of participating staff (e.g., physicians, nurses, and specialists), who are simultaneously moving in a constricted space, representing an additional burden on the motor apparatus of the participating staff. The aim is the most ergonomic possible arrangement for all participating staff, but this is difficult to implement. Capturing and analyzing clinical workflows by cameras is familiar from the prior art.

The publication “Rückengerechtes Arbeiten im OP” {“Back-friendly working in the operating theater”} by A. and T. Ammann, Schlütersche Verlagsgesellschaft mbH & Company KG, 2013, reports how the co-workers in an operating theater are exposed to an enormous load on the muscular and skeletal system, and that it is important to make the co-workers correspondingly mindful of paying attention to their motor and support apparatus. This should occur as a matter of the individual's own responsibility.

### BRIEF SUMMARY AND DESCRIPTION

The scope of the present disclosure is defined solely by the appended claims and is not affected to any degree by the statements within this summary. The present embodiments may obviate one or more of the drawbacks or limitations in the related art.

Embodiments provide a method, which enables an improvement in the ergonomics for staff in hospitals, for example operating theaters.

The method for automatic actuation of at least one device in the medical environment includes the following steps: recording of at least one image in a medical environment with an arrangement in which at least one person of the medical staff and at least one device that is adjustable or movable by motorized mechanisms are present, segmentation of the image and identification of the at least one person and/or the at least one adjustable or movable device, evaluation of the arrangement in the image in terms of at least one ergonomic parameter with regard to the at least one person, determination of an adjustment or a movement of the at least one device, that improves the ergonomic parameter with regard to the at least one person, and (automatic) actuation of the device for autonomous adjustment or movement of the device such that the ergonomic parameter is improved.

The arrangement in the medical environment may be formed by a scenario in an operating theater or examination room before or during an operation or examination with medical staff, that is to say e.g., physician, nurse, technician, or member of ancillary staff. The adjustable or movable device may include for example of a motor-adjustable (e.g., height adjustable) table and/or a movable trolley and/or a movable equipment trolley and/or a (mobile or permanently installed) medical apparatus and/or a monitor trolley and/or a waste container and/or a seating device (chair, seat) and/or a step. The devices are configured to be adjustable or movable by motorized mechanisms and may be adjusted or moved under actuation by a system controller for example. An arrangement with the at least one person and the at least one device (e.g., a nurse at an adjustable table with operating instruments, a doctor during the operation on the patient etc.) is for example recorded by a camera, segmented, and evaluated in terms of the ergonomic situation for the person. For example, the evaluation includes whether the person is standing bent or crooked or is in an otherwise non-optimal position or posture (that is to say e.g., for the skeleton or the motor apparatus). Then an adjustment option or a movement option is determined that results in an improvement in the ergonomic situation for the person. The adjustment or the movement of the device is then carried out automatically.

Such an analysis of an arrangement in the operating theater in terms of ergonomic parameters for a person, and an automatic adjustment of devices, makes it possible to improve the ergonomic situation for the medical staff simply, rapidly, and flexibly. The persons participating do not need to take action themselves and may concentrate fully on their tasks to continue the operation or their activity. As a result of the method, they are automatically alerted to an improvement in their ergonomic situation, and this is implemented. The method results in a substantial improvement in the health burden on, and working situation of, medical staff and serves to prevent long-term health problems.

According to an embodiment a series of images is recorded, segmented, and evaluated with a, for example changing, arrangement, and an adjustment or a movement is determined. As a result of recording more than one image a working situation in an operating theater may be analyzed even more effectively and e.g., work processes that are ergonomically problematical may also be included in the evaluation.

Likewise, it is advantageous to record at least two persons in the image or series of images. Accordingly, the arrangement may then be evaluated advantageously in terms of at least one ergonomic parameter for each of the at least two persons, and an adjustment or a movement of the at least one device may be determined, which improves the ergonomic parameters with regard to the at least two persons. Thus, an improvement in the ergonomic situation may be implemented in the case of multiple persons simultaneously.

According to a further embodiment at least two adjustable or moveable devices are present in the arrangement, and correspondingly the recorded images. For the devices options for adjustment or movement, that improve the ergonomics with regard to the person(s) of the medical staff, may then be determined simultaneously.

According to an embodiment the ergonomic parameter takes account of the skeleton and/or body posture of the person. Thus e.g., the skeleton or body posture of the person may be determined from the image or images, and on this basis a harmful posture identified, for example by determining a bending angle of the skeleton or a divergence from a body posture defined as healthy. Then an adjustment of the device is determined, that improves or entirely prevents a harmful posture of the skeleton or body of the person. Time-based factors may also be included from image series, for example the person remaining too long in the same position or a physically demanding movement.

According to a further embodiment the ergonomic parameter takes account of the walking route of the person. Thus e.g., a walking route analysis may be carried out and subsequently an adjustment or a change of position of the device carried out, to improve the walking route of the person, e.g., by avoiding detours. For example, a device located in the walking route of the person may be moved aside to make work easier for the person.

According to an embodiment one or more steps of the method may be carried out by at least one algorithm for machine learning (for example, using deep learning). Thus e.g., an algorithm may be trained to evaluate the arrangement of the image in terms of at least one ergonomic parameter or to determine an adjustment or a movement of the device, that improves the ergonomic parameter with regard to the at least one person. Such algorithms may carry out the steps rapidly and effectively after corresponding training. They are also suitable for ensuring implementation of the steps in real time in the operating theater.

According to a further embodiment a display or warning linked to the ergonomic parameter is output. Thus, it is possible to output to the person, for example on a monitor or some other display, that (and to what extent) their ergonomic situation (e.g., posture, position, table height, walking route) is unfavorable or harmful. An optical, acoustic, or haptic warning may also be output. This may be done with multiple persons.

According to a further embodiment an instruction is output, which improves the ergonomic parameter when carried out. For example, a suggestion for an improvement in the ergonomic situation may be output, for example a suggestion for an improved posture or a request to change posture regularly oneself. In the case of unfavorable walking routes e.g., alternative routes may be suggested, or in the case of multiple persons mutually impeding each other improvement suggestions may be provided for all participating persons. The output may be e.g., optical (display), acoustic, or haptic.

Additionally embodiments provide an overall system for carrying out a method as described above, including an image recording device, for example a camera, for recording at least one image, at least one adjustable or movable device, a system controller for actuation of the device, an image editing unit for segmenting the image and for identifying persons and/or devices, an evaluation unit for evaluating the image in terms of at least one ergonomic value with regard to the at least one person, and a processing unit for determining an adjustment or a movement of the device, that improves the ergonomic value for the at least one person. According to a variant the overall system includes at least one algorithm for machine learning, that carries out evaluation of the image in terms of at least one ergonomic value, and/or determination of an adjustment or a movement of the device, which improves the ergonomic parameter with regard to the at least one person. Additionally, a display unit and a communication unit may also be present.

### BRIEF DESCRIPTION OF THE FIGURES

FIG. 1 depicts a sequence of steps in a method for automatic actuation of at least one device in the medical environment according to an embodiment.

FIG. 2 depicts a view of an overall system for carrying out the method according to an embodiment.

FIG. 3 depicts a view of an incorrect posture of a person.

### DETAILED DESCRIPTION

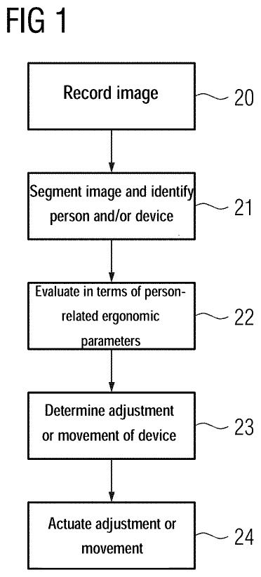

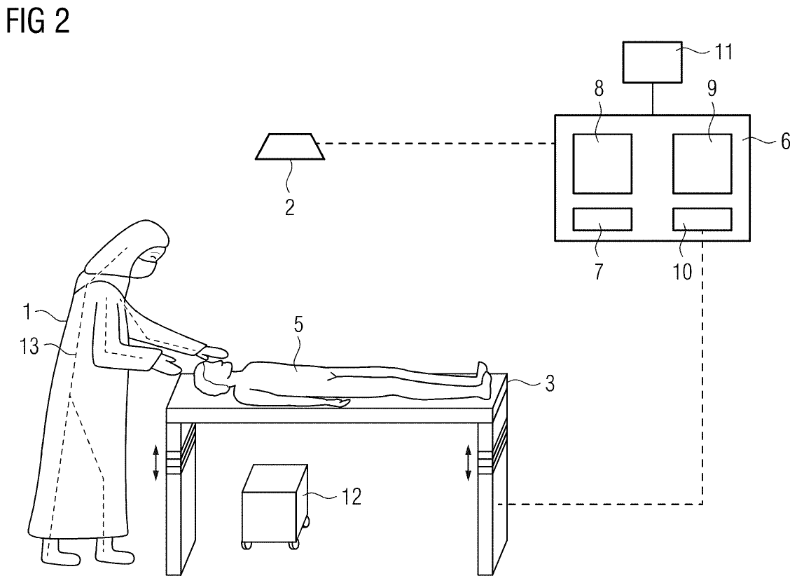

The method and overall system claimed is suitable for ensuring the healthiest possible working conditions for hospital staff and a particularly smooth process in clinics and operating theaters. FIG. 1 depicts a sequence of steps in a method. FIG. 2 presents an overall system for carrying out the method.

In a first step 20 at least one image or one scenario is recorded in a medical environment with an arrangement in which at least one person 1 of the medical staff and at least one device that is adjustable or movable by motorized mechanisms, for example a height-adjustable patient table 3, is present—see FIG. 2. The recording is carried out for example by a camera 2, for example a 3D camera. Other image recording devices may also be used. Multiple images or a (time) sequence of images may also be recorded. The arrangement in the medical environment may be formed by a situation in an operating theater before or during an operation or examination with medical staff. The person 1 of the medical staff may include of e.g., a physician, a nurse, a technician, or a member of ancillary staff. The adjustable or movable device may include, for example, a motor-adjustable (e.g. height adjustable) patient table 3 and/or a movable trolley 12 and/or a movable equipment trolley (e.g. anesthesia trolley) and/or a (mobile or permanently installed) medical apparatus (e.g. X-ray device, ultrasound device, etc.) and/or a monitor trolley and/or a waste container and/or a seating device (chair, seat) and/or a step and/or another device. The devices are configured to be adjustable or movable by motorized mechanisms and may be adjusted or moved under actuation by a system controller 6 for example. Multiple persons and multiple devices may also be recorded.

In a second step 21 the recorded image is segmented and the person 1 and the adjustable or movable device, for example a patient table 3, are identified in the image, for example by segmentation and object recognition software on an image editing unit 7. Where there are multiple persons and/or multiple devices present the multiple persons and/or multiple devices may be correspondingly segmented and identified.

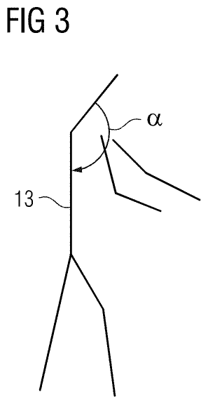

In a third step 22 an evaluation of the images (or images) takes place in terms of at least one ergonomic parameter relating to the at least one person 1. The evaluation in terms of an ergonomic parameter may contain quite generally an appraisal of the ergonomic situation of the person, or a specific assessment of a partial aspect of the ergonomics in relation to one or more devices. Thus, it may provide a simple breakdown in to “good”, “moderate”, and “poor”, or e.g., in the case of specific aspects contain a measured value. Thus, for example it is possible to evaluate whether the person is adopting a static incorrect posture (e.g., standing too bent over or in a crooked manner) or is in some other unsuitable position or posture in health terms (that is to say e.g., for the skeleton 13 or the motor apparatus). Thus, for example the ergonomic parameter may relate to the skeleton and/or the body posture of the person. The skeleton or body posture of the person may be determined e.g., from the image section segmented and identified as a person. Based on this a harmful posture/position may then be identified, for example by determining a bending angle of the cervical spinal column of the person 1 (see FIG. 3) and/or a divergence from a body posture defined as healthy for the person. In the case of multiple persons this may be carried out for all the identified persons. Alternatively, a prioritization or selection of persons may also be made. Time-based ergonomic parameters may also be brought in from multiple consecutive images or image series, for example the person remaining too long in the same position or a physically demanding movement. As a result, e.g., work processes that are ergonomically problematical may also be included in the evaluation; for example, a walking route analysis may also be carried out. The evaluation is carried out e.g., by an evaluation unit 8.

In a fourth step 23 an adjustment or a movement of the at least one device is then determined or calculated, e.g., by a processing unit 9, that improves the ergonomic parameter of the person or the ergonomic parameters of the persons for a short or longer time. Thus, for example in the case of the height-adjustable table 3 different table positions may be determined to improve the ergonomic parameter (that is to say e.g., the body posture, bending of the back, hand position, etc.) of the person standing at the table.

Adjustment options may also be determined for two or more devices. An improvement in the ergonomic situation may be implemented for multiple persons at the same time. With regard to the devices, it is then possible to simultaneously determine options for adjustment or movement, that improve the ergonomics with regard to the person(s) of the medical staff. It is also possible to carry out, from an image sequence over time, e.g., a walking route analysis and then carry out an adjustment or a change of position of the device to improve the walking route of the person, e.g., by avoiding detours. For example, a new position may be determined for a device located on the walking route of the person. Additionally, adjustment options that are coordinated with each other over time may also be determined.

One or more steps in the method may be carried out by at least one algorithm for machine learning (for example deep learning), for example the third step 22 or the fourth step 23. Thus, for example a machine learning algorithm may be trained to evaluate the arrangement of the image in terms of at least one ergonomic parameter or to determine an adjustment or a movement of the device, that improves the ergonomic parameter with regard to the at least one person. Algorithms of this type may carry out the steps after corresponding training.

Actuation for the corresponding adjustment or movement of the device to improve the ergonomic parameter is then carried out automatically in a fifth step 24, for example with the adjustable table 3 being actuated by the system controller 6 to take up a different table height. With regard to actuation of the camera 2 and/or the device the overall system may also include a communication unit 10. The overall system may also be assigned to e.g., a medical imaging system (e.g., X-ray device, ultrasound device, MR, etc.) or be integrated in to same.

The method may be carried out e.g., live and in real time in examination rooms or operating theaters, e.g., during a surgical operation, during long-lasting examinations, or also during preparations for same.

In addition to an automatic improvement a display or warning linked to the ergonomic parameter may also be output. Thus, for example it is possible to output to the person, for example on a monitor 11 or some other display, that (and to what extent) their ergonomic situation (e.g., posture, position, table height, walking route) is unfavorable or harmful and/or how improvement may be made by the automatic adjustment. A general optical, acoustic, or haptic warning may also be output, e.g., that an adjustment is currently taking place.

Additionally, an instruction may be output, which (further) improves the ergonomic parameter when carried out, for example a suggestion for an improved posture or a request to change posture regularly oneself. In the case of unfavorable walking routes e.g., alternative routes or alternative positions for objects may be suggested or in the case of multiple persons mutually impeding each other improvement suggestions may be provided for all participating persons. With regard to time-related correction in the case of multiple persons suggestions for defined start times or sequences of persons may also be suggested to minimize mutual impeding/collision or prevent the coming together of sterile and non-sterile persons.

A further example of an application may also be a person belonging to the medical staff who is standing at a height-adjustable or movable instrument table and handing instruments to the doctor in the operating theater.

Embodiments provide a good ergonomic working atmosphere of the medical staff during examinations and operations including where a method for automatic actuation of at least one device in the medical environment is provided with the following steps: recording of at least one image in a medical environment with an arrangement in which at least one person of the medical staff and at least one device that is adjustable or movable by motorized mechanisms are present, segmentation of the image and identification of the at least one person and/or the at least one adjustable or movable device, evaluation of the arrangement in the image in terms of at least one ergonomic parameter with regard to the at least one person, determination of an adjustment or a movement of the at least one device, that improves the ergonomic parameter with regard to the at least one person, and (automatic) actuation of the device for autonomous adjustment or movement of the device such that the ergonomic parameter is improved.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for automatic actuation of at least one device that is adjustable or movable by motorized mechanisms in a medical environment, the method comprising:
recording at least one image in the medical environment with an arrangement in which at least one person of a medical staff and the at least one device are present;
segmenting the at least one image and identifying the at least one person or the at least one device;
evaluating the arrangement in the image in terms of at least one ergonomic parameter with regard to the at least one person;
determining an adjustment or a movement of the at least one device that improves the ergonomic parameter with regard to the at least one person; and
automatically actuating the at least one device for autonomous adjustment or movement of the at least one device such that the ergonomic parameter is improved.

2. The method of claim 1, wherein a series of images is recorded, segmented, and evaluated with a changing arrangement, wherein the adjustment or the movement is determined based on the evaluation of the changing arrangement.

3. The method of claim 1, wherein at least two persons are recorded.

4. The method of claim 3, wherein the arrangement is evaluated in terms of at least one ergonomic parameter for each of the at least two persons, and an adjustment or a movement of the at least one device is determined that improves the ergonomic parameters with regard to the at least two persons.

5. The method of claim 1, wherein at least two adjustable or moveable devices are present.

6. The method of claim 1, wherein the at least one ergonomic parameter is based on a skeleton, body posture, or skeleton and body posture of the at least one person.

7. The method of claim 1, wherein the at least one ergonomic parameter is based on a walking route of the at least one person.

8. The method of claim 1, wherein evaluating the arrangement is carried out by at least one algorithm for machine learning.

9. The method of claim 1, further comprising:
outputting a display or warning linked to the ergonomic parameter.

10. The method of claim 1, further comprising:
outputting an instruction that improves the ergonomic parameter when carried out.

11. The method of claim 1, wherein the at least one device comprises at least one of a table, a trolley, an equipment trolley, a medical device, a monitor trolley, a waste container, a seating device, or a step.

12. A system for automatic actuation of at least one device in a medical environment, the system comprising:
an image recording device configured to record at least one image of at least one person and at least one device;
the at least one device that is adjustable or movable by motorized mechanisms;
a system controller configured for automatic actuation of the at least one device;
an image editing unit configured to segment the at least one image and to identify the at least one person and the at least one device; and
an evaluation unit configured to evaluate the at least one image in terms of at least one ergonomic parameter with regard to the at least one person and at least one device, the evaluation unit further configured to determine an adjustment or a movement of the at least one device that improves the ergonomic parameter for the at least one person.

13. The system of claim 12, wherein the evaluation unit includes at least one algorithm for machine learning configured to carry out the evaluation or the determination.

14. A non-transitory computer readable storage medium comprising a set of computer-readable instructions stored thereon for automatic actuation of at least one device that is adjustable or movable by motorized mechanisms in a medical environment, the instructions which, when executed by at least one processor cause the processor to:
record at least one image in the medical environment with an arrangement in which at least one person of a medical staff and at least one device are present;
segment the at least one image and identifying the at least one person or the at least one adjustable or movable device;
evaluate the arrangement in the image in terms of at least one ergonomic parameter with regard to the at least one person;
determine an adjustment or a movement of the at least one device that improves the ergonomic parameter with regard to the at least one person; and
automatically actuate the at least one device for autonomous adjustment or movement of the at least one device such that the ergonomic parameter is improved.

15. The non-transitory computer readable storage medium of claim 14, wherein a series of images is recorded, segmented, and evaluated with a changing arrangement, wherein the adjustment or the movement is determined based on the evaluation of the changing arrangement.

16. The non-transitory computer readable storage medium of claim 14, wherein at least two persons are recorded.

17. The non-transitory computer readable storage medium of claim 16, wherein the arrangement is evaluated in terms of at least one ergonomic parameter for each of the at least two persons, and an adjustment or a movement of the at least one device is determined that improves the ergonomic parameters with regard to the at least two persons.

18. The non-transitory computer readable storage medium of claim 14, further comprising instructions to output a display or warning linked to the ergonomic parameter.

19. The non-transitory computer readable storage medium of claim 14, wherein the at least one ergonomic parameter is based on a skeleton, body posture, or skeleton and body posture of the at least one person.

20. The non-transitory computer readable storage medium of claim 14, wherein evaluating the arrangement is carried out by at least one algorithm for machine learning.
